(19) United States
(12) Patent Application Publication
(10) Pub. No.: US 2013/0210063 A1
(43) Pub. Date: Aug. 15, 2013

(54) COMPOSITIONS AND ASSAYS FOR DETERMINING CELL VIABILITY

(71) Applicant: Bonnie Jean Hanson, Madison, WI (US)

(72) Inventor: Bonnie Jean Hanson, Madison, WI (US)

(73) Assignee: LIFE TECHNOLOGIES CORPORATION, Carlsbad, CA (US)

(21) Appl. No.: 13/879,621

(22) PCT Filed: Oct. 13, 2011
(86) PCT No.: PCT/US11/56186

§ 371 Date: Apr. 15, 2013

Related U.S. Application Data
(60) Provisional application No. 61/392,922, filed on Oct. 13, 2010.

(51) Int. Cl. C12Q 1/02; G01N 33/50; C12Q 1/18

(52) U.S. Cl. CPC C12Q 1/02; C12Q 1/025; C12Q 1/18; G01N 33/5014; G01N 33/5005
USPC 435/32; 435/29

(57) ABSTRACT

Provided are compositions and methods useful in evaluation of cell health and metabolism, cell viability, proliferation, and the effects of compounds on these qualities. The assays provided are rapid, robust, nontoxic and suitable for use with high throughput devices.

### CROSS REFERENCE TO RELATED APPLICATIONS

This application claims the benefit under 35 U.S.C. 119(e) of U.S. Provisional Patent Application No. 61/392,922, filed Oct. 13, 2010, which is hereby incorporated by reference.

### TECHNICAL FIELD

The present invention relates to compositions, methods and kits for assessing cell metabolic activity, viability, and proliferation. This invention also relates to compositions, methods and kits for determining the susceptibility of cells to cytotoxic and growth inhibitory agents or conditions.

### BACKGROUND

Determination of cell viability, metabolic activity, and/or cell proliferation is important in a wide range of applied and research situations. Methods for determining cell viability or cell cytotoxicity in response to exposure to test agents or conditions are important to pharmaceutical and environmental testing, pesticide and herbicide testing and drug discovery. Methods for determining cell proliferation are of widespread use in basic research, as well as food safety testing and bioproduction of regulated biologics. A method that reliably and accurately measures cell viability and/or toxicity after exposure to a test agent is important in determining whether a particular chemical agent presents a real or potential risk when exposed to a given cell type.

A variety of means and methods have been used to determine cell viability. For example, some methods are based on the ability of the membrane of viable cells to exclude vital dyes such as trypan blue, propidium iodide, and ethidium monoazide. Living cells exclude such vital dyes whereas dead or dying cells that have lost membrane integrity permit entry of these dyes into the cytoplasm, where the dyes stain various compounds or organelles within the cell. Non-viable cells that have lost membrane integrity also leak cytoplasmic components into the surrounding medium. Cell death thus can be measured by monitoring the concentration of these cellular components in the surrounding medium. For example, the release of glycerldehyde-3-phosphate dehydrogenase (G3PDH) from dead or damaged cells is measured by coupling the activity of the released G3PDH to the production of ATP (Corey et al. (1997) J. Immunol. Meth. 207:43-51).

Other methods to test for cell viability, metabolic activity, cell proliferation, or cell death rely upon the conversion of a dye from one state to another. For example, in a typical format, prior to the reaction the dye absorbs at a first wavelength of radiation. The dye is then converted to a product that absorbs at a second (and different) wavelength of light. By monitoring the conversion of the dye from one state to the other, the extent of cell viability or cell death can be determined A number of suitable dyes for this purpose are known, and of these indicators, electron-acceptor dyes such as tetrazolium salts are frequently used. Tetrazolium salts known in the art include MTT (3-(4,5-dimethylthiazol-2-yl)-2,5-diphenyltetrazolium bromide), XTT (sodium 3′-{(1-phenylamino-carbonyl)-3,4-tetrazolium}-bis(4-methoxy-6-nit-ro)benzene-sulfonic acid hydrate), and MTS (3-(4,5-dimethylthiazol-2-yl)-5-(3-carboxymethoxyphenyl)-2-(4-sulphophenyl-)-2H-tetrazolium, inner salt). See, for example, van de Loosdrecht et al. (1994) J. Immunol. Methods 174:311-320; Buttke et. al. (1993) J. Immunol. Methods 157: 233-240; Berridge et al. (2005) Biotechnol. Annu. Rev. 11:127-152.

Resazurin is a non-toxic, cell permeable compound that, in its oxidized state, is blue in color and virtually non-fluorescent. In living cells, resazurin is reduced to resorufin, a compound that is red in color and highly fluorescent, and can be detected fluorimetrically or colorimetrically. Metabolic activity of viable cells continuously convert resazurin to resorufin, increasing the overall fluorescence and color of the media surrounding cells. O'Brien et al. (2000) Eur. J. Biochem. 267:5421-5426.

Historically, resazurin has been used in the dairy industry to monitor microbial contamination in milk, and also for assessing semen quality. The use of absorbent pads impregnated with resazurin and antibiotics for antimicrobial susceptibility testing are described in Baker et al. (1980) Microbiol. 26:248-253 and Canadian Patent No. 1,112,140. Bacterial isolates are applied to the pad in a brain heart infusion broth. Kanazawa et al. (1966) J. Antibiotics 19:229-233 also describe the use of absorbent pads impregnated with resazurin and antimicrobial agents for use in susceptibility testing. The protocols described, however, are not suitable, for example, for determining minimum inhibitory concentrations (MIC).

U.S. Pat. No. 5,501,959 describes a cell viability and proliferation assay wherein microorganisms, tissue cells, or the like, are incubated in a growth medium in the presence of the dye resazurin and a compound to be tested. A redox stabilizing agent, called a “poising” agent, is also added to the reaction mix to inhibit non-specific autoreduction of the resazurin due to components found within most culture media. In this assay, the resazurin dye is reduced by the activity of living cells and is used as a redox indicator to detect microbial cell growth or mammalian cell growth.

A number of cell viability assays and cytotoxicity assays using resazurin are commercially available. For example, alamarBlue® cell viability reagent (Life Technologies Corporation, Carlsbad, Calif.) is based on the technology of U.S. Pat. No. 5,501,959. The alamarBlue® product literature instructs incubation of cells in dye reagent for 1-4 hours, or longer for greater sensitivity, before measuring resorufin fluorescence or absorbance. Promega Corporation (Madison, Wis.) markets CellTiter-Blue® Cell Viability Assay which uses a buffered solution containing highly purified resazurin. The CellTiter-Blue® product literature recommends incubation of cells in the dye reagent for 1-4 hours, or longer for increased sensitivity, before fluorescence or absorbance is measured. TOX-8 In Vitro Toxicology Assay Kit (Sigma-Aldrich Co., St. Louis, Mo.) is designed for fluorometrically or spectrophotometrically determining cell number as a function of metabolic activity using the dye resazurin. The TOX-8 product literature instructs incubation of cells in the resazurin dye solution for 2-4 hours or longer before the dye conversion is measured.

Life Technologies Corporation markets Vybrant™ Cell Metabolic Assay Kit which uses lipophilic C₁₂-resazurin. This modified resazurin exhibits enhanced cellular retention compared with alamarBlue® reagent. Life Technologies Corporation also markets Vybrant™ Cytotoxicity Assay Kit which contains all of the enzymes and substrates needed to monitor the release of the cytoplasmic enzyme glucose-6-phosphate dehydrogenase (G6PD) from damaged or dying cells into the surrounding medium. This assay detects G6PD via a two-step process that leads to the reduction of resazurin to resorufin. G-Biosciences (Maryland Heights, Mo.) markets CytoScan™-fluoro Cytotoxicity Assay which detects the release of lactate dehydrogenase (LDH) from damaged or stressed cells. This assay detects LDH via a two-step process that leads to the reduction of resazurin to resorufin.

Cell viability and cytotoxicity assays based on detection of release of LDH by dead cells but not involving resazurin are also commercially available. For example, Promega Corporation markets CytoTox 96® Assay, Sigma-Aldrich markets Tox-7 In Vitro Toxicology Assay Kit, and G-Biosciences markets CytoScan™ LDH Cytotoxicity Assay.

There remains, however, a need for an assay for cell metabolic activity, viability, proliferation, and/or cytotoxicity that is, for example, rapid, comprises components that are non-toxic (allowing cells to remain viable through and after the test), and that is amenable to high throughput screening.

All patents, patent applications, and publications cited herein are hereby incorporated herein by reference in their entirety.

### SUMMARY

This invention provides, in part, methods for assessing cell viability, metabolic activity, cell proliferation, and cytotoxicity. In one aspect, the invention provides a method for assessing a metabolic activity of a cell in a cell culture. The method comprises adding a reagent solution comprising resazurin, a buffer and an aqueous solvent to the cell culture, wherein the resazurin is initially present in the oxidized state, and measuring for production of the reduced state of the resazurin within 60 minutes of adding the reagent solution to the cell culture for the assessing of metabolic activity. In some embodiments, the cell is suitable for another functional assay following the assessing for metabolic activity.

In another aspect, the invention provides a method for determining cytotoxicity of a test agent comprising: (a) contacting a living cell in a culture medium with the test agent and incubating the cell and the test agent in the culture medium for an amount of time; (b) adding to the cell and the culture medium from (a) a reagent solution comprising resazurin, a buffer and an aqueous solvent, wherein the resazurin is initially present in an oxidized state; and (c) measuring for production of a reduced state of the resazurin from (b) within 60 minutes of addition of the reagent solution, wherein the measuring indicates the cytotoxicity of the test agent. In some embodiments, the cell is suitable for a functional assay following the measuring in (c). In some embodiments, the contacting and the incubating in (a), the adding of the reagent solution in (b) and the measuring in (c) are all performed in the same vessel.

In some embodiments of the invention, measuring for the production of the reduced state of the resazurin involves fluorescent spectroscopy or visible spectroscopy. In some embodiments, the measuring is performed within 30 minutes of adding the reagent solution. In other embodiments, the measuring is performed about 20 minutes or about 10 minutes after adding the reagent solution.

In some embodiments, the measuring is performed without separating the culture medium from the cell. In some embodiments, the adding of the reagent solution and the measuring for production of the reduced state of resazurin are both performed in the same vessel.

In some embodiments, the cell is prokaryotic, for example, a bacterial cell. In other embodiments, the cell is eukaryotic, for example a mammalian cell or an insect cell. In some embodiments, the cell is a primary cell. In other embodiments, the cell is a cultured cell. In some embodiments, the cell is grown in suspension culture, monolayer culture, adherent culture or on an absorptive disk.

In some embodiments, the buffer in the reagent solution is a phosphate buffer, a Tris buffer, HEPES, or MOPS. In some embodiments, the reagent solution comprises about 0.1 M to about 0.3 M phosphate buffer. In some embodiments, the reagent solution comprises from about 0.35 mM to about 0.60 mM resazurin. In some embodiments, the reagent solution buffer has a pH of from about 7.0 to about 7.8. In certain embodiments, the reagent solution comprises: 0.35 mM to about 0.60 mM resazurin, and about 0.1 M to about 0.3 M phosphate buffer at a pH from about 7.2 to about 7.5.

In some embodiments, test agents are known or putative antibiotics, known or putative pharmaceutical or therapeutic agent, or known or putative toxic compounds.

Another embodiment of the invention provides a method for determining cytotoxicity of a test agent comprising: (a) contacting a living cell in a culture medium with a pre-determined amount of the test agent and incubating the cell and the test agent in the culture medium for a pre-determined amount of time; (b) adding to the cell and the culture medium from (a) a reagent solution comprising resazurin, a buffer and an aqueous solvent; and (c) measuring for production of resorufin within 60 minutes of addition of the reagent solution, wherein the measuring indicates cytotoxicity of the test agent.

Another embodiment of the invention provides a method for determining cytotoxicity of a test agent comprising: (a) contacting a living cell in a culture medium with the test agent and incubating the cell and the test agent in the culture medium for an amount of time; (b) adding to the cell and the culture medium from (a) a reagent solution consisting essentially of resazurin, a buffer and an aqueous solvent, wherein the resazurin is initially present in an oxidized state; and (c) measuring for production of a reduced state of the resazurin from (b), wherein the measuring indicates cytotoxicity of the test agent.

Another embodiment of the invention provides a method for determining cytotoxic effect of a test agent on growth of cultured cells comprising: (a) culturing the cells in a growth medium in the presence of the test agent for an amount of time; (b) adding to the cells and the growth medium from (a) a reagent solution comprising resazurin, a buffer and an aqueous solvent; and (c) measuring for production of resorufin within 60 minutes of the addition of the reagent solution in (b), wherein the measuring indicates the cytotoxic effect of the test agent on growth of the cells.

Another embodiment of the invention provides a method for determining cytotoxicity of a test culture condition comprising: (a) culturing living cells in a culture medium under a test culture condition for an amount of time; (b) adding to the cells and the culture medium from (a) a reagent solution comprising resazurin, a buffer and an aqueous solvent, wherein the resazurin is initially present in an oxidized state; and (c) measuring for production of a reduced state of the resazurin from (b) within 60 minutes of the addition of the reagent solution, wherein the measuring indicates the cytotoxicity of the test culture condition.

In another aspect, the invention provides a kit for assessing metabolic activity of a cell comprising a reagent solution disposed in a container. In some embodiments, the reagent solution of the kit consists essentially of an aqueous solvent, from about 0.35 mM to about 0.60 mM resazurin, and about 0.1 M to about 0.3 M phosphate buffer at a pH from about 7.2 to about 7.5.

### BRIEF DESCRIPTION OF THE DRAWINGS

FIG. 1 is a graph showing fluorescence results of a 5 minute incubation of cells with the indicator dyes: Reagent Solution (circle), alamarBlue (square) and CellTiter-Blue (diamond). RFU, relative fluorescence units.

FIGS. 2A-2F are graphs showing fluorescence results of varying incubation times of cells with dyes Reagent Solution (triangle) and alamarBlue (square). Incubation times: 10 minutes, FIG. 2A; 20 minutes, FIG. 2B; 1 hour, FIG. 2C; 2 hours, FIG. 2D; 4 hours, FIG. 2E; 24 hours, FIG. 2F.

FIG. 3 is graph showing results of a rapid assay with Reagent Solution (triangle) and alamarBlue (AB, square) with varying densities of Jurkat cells. RFU, relative fluorescence units

FIGS. 4A and 4B are graphs showing results of rapid format assays with reagent solutions made with varying phosphate buffer concentrations and with alamarBlue (AB).

FIG. 4A shows results following a 6 minute incubation in reagent solution with buffer concentrations ranging from 0.05M to 1.0 M. FIG. 4B show results following a 15 minute incubation in reagent solution with buffer concentrations ranging from 0.08M to 0.5 M. RFU, relative fluorescence units.

FIGS. 5A and 5B are graphs showing results of extended time format assays with reagent solutions made with varying phosphate buffer concentrations and with alamarBlue (AB). FIG. 5A shows results following a 40 hour incubation in reagent solution with buffer concentrations ranging from 0.08M to 0.5 M. FIG. 5B show results following a 65 hour incubation in reagent solution with buffer concentrations ranging from 0.05M to 1.0 M. RFU, relative fluorescence units.

FIG. 6 is a graph showing results of a rapid assay format with reagent solutions made with 0.15 M phosphate buffer with varying pH values and with alamarBlue (AB). RFU ratio, relative fluorescence units of wells with cells/relative fluorescence units of control wells without cells.

FIG. 7 is a graph showing results of a rapid assay format with reagent solutions made with varying concentrations of resazurin and with alamarBlue (AB). RFU, relative fluorescence units.

FIGS. 8A-8C are graphs showing results of a rapid assay format with reagent solutions made with varying buffers and buffer concentrations (1M, inverted triangles; 0.15M diamonds; 0.025 M, circles), with a standard reagent solution (triangles) and with alamarBlue (squares). FIG. 8A shows results with reagent solutions made with Tris buffer, FIG. 8B shows results with reagent solutions made with MOPS buffer, and FIG. 8C shows results with reagent solutions made with HEPES buffer.

FIGS. 9A-9D are graphs showing results of cytotoxicity assays using etoposide (squares), doxorubicin (solid triangles), chetomin (inverted triangles) and staurosporine (open triangles) and different indicator dyes : Reagent Solution (FIG. 9A), alamarBlue (FIG. 9B), CellTiter-Blue (FIG. 9C), and CellTiter-Glo (FIG. 9D). RFU, relative fluorescence units. EC50 calculations for each compound is below each graph.

FIG. 10 is a graph showing results of a rapid assay format with varying densities of cells incubated with the reagent solution at 25° C. (stippled bar) or 37° C. (solid bar) for 10 minutes.

FIG. 11 is a graph showing signal results from an assay with the reagent solution before (diamonds) and 24 hours after (squares) addition of SDS to the assay cells.

FIG. 12 is a graph showing fluorescence results of assays varying cell densities of E. coli cells incubated with reagent solution for 15 minutes (squares), 30 minutes (triangles) or 1 hour (inverted triangles).

FIG. 13 is a graph showing fluorescence results of assays of varying cell densities of Sf9 insect cells incubated with reagent solution for 10 minutes (squares) or 1 hour (triangles).

### DETAILED DESCRIPTION

The present invention relates, in part, to cell viability, metabolic activity, cell proliferation, and cytotoxicity assays which are rapid, robust, nontoxic and suitable for use with high throughput/robotic devices. Applicants have discovered resazurin-based reagent solutions that provide cell viability and metabolic activity measurements in a rapid assay format. As demonstrated herein, the reagent solutions of the invention allow a significantly reduced incubation time prior to assay read-out compared to currently commercially-available resazurin-based assays. Commercially-available resazurin-based assays recommend a 1-4 hour (or longer) incubation time of the cells with the resazurin dye solution prior to measuring the resultant fluorescence or absorbance. With the assay methods and compositions provided herein, this incubation time can be 10 minutes or less. Also as demonstrated herein, the signal-to-background with the provided compositions is larger than other commercially available resazurin-based products.

In one embodiment of the instant invention, metabolically active cells reduce the virtually non-fluorescent resazurin to resorufin, a highly fluorescent compound. Resorufin also has a different absorbance wavelength maximum than resazurin, allowing the assay to alternatively be read out by absorbance. Nonviable cells rapidly lose metabolic activity and do not reduce resazurin to resorufin. The assay can be utilized to evaluate cell health and metabolism, determine cell viability and proliferation, and the effects of compounds on these qualities. In some embodiments, the assay provided herein is a homogeneous, fluorometric and/or colorimetric, rapid format assay for estimating the number of viable cells present in a cell culture flask, plate, or multiwell plate.

Certain non-resazurin assays in the cell health field that have 10 minute incubation periods, require cell lysis to read-out the assay results whereas the assays and reagent solutions of the instant invention do not require the cells to be lysed in order to gather the results in 10 minutes. Accordingly, cells can be cultured after exposure to the reagent solution in the provided assays. Such cells can be used for functional assays following the fluorometric and/or colorimetric measurement. Assays of the invention can thus be multiplexed with other cell health indicator assays or tests.

In addition to a rapid format without the requirement for cell lysis, the reagent solutions of the invention have a number of other advantages compared to commercially-available cell viability reagents, including, but not limited to: (1) good stability at wide range of temperatures, (2) suitable for a completely ready-to-use reagent (e.g., no need to thaw, mix together components, and provide special storage conditions), (3) homogeneous single-step addition only assay format, and (4) no need to mix before assay measurement (vs. mixing recommendation for viability assays that involve a cell lysis step for best Z′ values), and (5) likely more amenable to high throughput screening (HTS)/robotic dispensing platforms than viability assays that present solution viscosity issues.

The reagent solution comprises, for example, an aqueous solvent (such as water), a buffer, and a dye having an oxidized state and a reduced state wherein the reduced state can be distinguished from the oxidized state (for example, resazurin). Resazurin, 7-hydroxy-3H-phenoxazin-3-one 10-oxide, is commercially available, for example as a sodium salt, from Sigma-Aldrich and other chemical suppliers.

The reagent solutions provided herein are non-toxic to living cells. Accordingly, the reagent solution can be added directly to the cells being assessed without adversely affecting the outcome of the assay. Since this is suitable for assaying live cells, readings may be taken at multiple time points during a given assay.

In one embodiment of the instant invention, cells are incubated for an amount of time in the presence of the test agent, after which time the resazurin containing reagent solution is added to the incubated cells. Metabolically active cells reduce the resazurin to resorufin and within 60 minutes of the addition of the resazurin to the cells, the resorufin is detected or measured colorimetrically or fluorimetrically. Suitable measuring devices are well known in the art and can be purchased from many commercial suppliers.

In one embodiment, cells are mixed with the reagent solution in a vessel. In some embodiments, incubation of cells with the reagent solution and measurement of the reduced resazurin are both performed in the same vessel. In some embodiments, incubation of the cells with a test agent or under test conditions, subsequent incubation of the cells with the reagent solution, and measurement of the reduced resazurin are all performed in the same vessel. As used herein, “vessel” indicates any container or holder wherein the methods disclosed herein can occur, including without limitation, single well containers, such as test tubes, flasks, plates, cuvettes, bioreactors, and multi-well containers such as microtiter plates of any configuration. “Vessel” also encompasses pads, patches, tapes, bandages, and the like, of any material construction, that are sufficiently absorbent to retain the cells and reagents needed to perform the subject method.

Resazurin reduction can be detected either by absorbance colorimetry or by fluorimetry. Resazurin in its oxidized state is deeply blue in color and is essentially non-fluorescent, depending upon its level of purity. Resorufin, the reduced form of resazurin, is red and very fluorescent. When using colorimetry, the reaction is monitored at wavelengths well known in the art to be an absorption maximum for resorufin (approximately 570 nm). Fluorescence measurements of resorufin are made by exciting at wavelengths well known in the art (approximately 530 to 560 nm) and measuring the emission spectrum (known in the art to have a maximum at about 590 nm). Because fluorometric detection is more sensitive than spectrophotometric detection, in certain embodiments of the methods, the production of resorufin is detected fluorimetrically. Suitable measuring devices would be well known to one of skill in the art. Many measuring devices can be purchased from commercial suppliers. Exemplary devices include, without limitation, fluorimeters, fluorescent and/or visible light plate readers, microscopes, spectrophotometers, imaging systems, high content imagers and flow cytomers.

The medium in which the cells are grown or held does not limit the functionality of the methods or composition provided herein, although in certain embodiments, non-reducing media are used to minimize any non-specific reduction of the dye. For microbial cultures, suitable media include, without limitation, Mueller-Hinton Broth, Luria broth, and trypticase soy broth. For mammalian cell cultures, suitable media include, without limitation, RPMI 1640, RPMI 1640 plus fetal bovine serum, Dulbecco's Modified Eagle Medium (D-MEM), McCoy's 5A Medium, Medium 106, Hanks' Balanced Salt Solution, Phosphate Buffered Solution. The mammalian cell culture mediums may or may not be supplemented with serum, such as for example fetal bovine serum or newborn calf serum, and growth factors and other culturing supplements known in the art, such as amino acids, antibiotics an antimicrobial agents. For insect cell cultures, suitable media include, without limitation, SF-900 III SFM Medium, SF-900 II SFM Medium, Schneider's Drosophila Medium, and IPL-41 Insect Medium.

The assay methods and compositions provided herein can be used to, for example, assess viability, metabolic activity, proliferation and/or the effects of compounds on these qualities of a wide variety of living cells.

In one aspect, the invention provides methods for assessing metabolic activity of a cell in a population of cells. The general protocol of the method of measuring metabolic activity proceeds as follows: the reagent solution is added to the cell sample(s) and, within 60 minutes of adding the reagent solution, production of the reduced state of resazurin is measured. Production of reduced resazurin is measured, for example, by gathering fluorescent or colorimetric data from the cell sample. Reduced resazurin is measured in the cells, in the media or in both the cells and the media. In some embodiments, the fluorescent or colorimetric data is gathered by measuring the media containing the cells. In some embodiments, the fluorescent or colorimetric data is gathered by measuring the media without the cells. In some embodiments, the fluorescent or colorimetric data is gathered by measuring within or on the cells.

In another aspect, the invention provides methods for determining cytotoxicity of a test agent. In another aspect methods for determining cytotoxicity of a test culture condition on a cell are provided. In another aspect, methods are provided for determining a cytotoxic effect of a test agent or a test culture condition on growth of cells are provided. A general protocol of the method of measuring cytotoxicity or a cytotoxic effect proceeds as follows: living cells in a medium are contacted with a test agent or placed under test conditions for an amount of time, the reagent solution is added to the cells, and, within 60 minutes of adding the reagent solution, production of the reduced state of resazurin is measured. In some embodiments, living cells in a medium are contacted with a test agent or placed under test conditions for a pre-determined amount of time prior to addition of the reagent solution. In some embodiments, living cells in a medium are contacted with a pre-determined amount of test agent for an amount of time prior to addition of the reagent solution.

In other embodiments, cytotoxicity of a test agent can be assessed in real time by contacting living cells in a medium with a test agent and the reagent solution at the same time, and, within 60 minutes of adding the test agent and reagent solution, measuring production of the reduced state of resazurin. In such real time assays, control wells in which cells were contacted with the reagent solution but with the test agent would be assessed at the same time to show cell viability in absence of the test agent.

The test agent may be an agent with a known activity or with a putative activity that is being investigated for a particular use. For example, a known or a putative pharmaceutical agent is a chemical compound or formulation known and used as a pharmaceutical agent, or a chemical compound or formulation being investigated for use as a pharmaceutical agent. Likewise, a known or a putative therapeutic agent is a chemical compound or formulation known and used as a therapeutic agent, or a chemical compound or formulation being investigated for use as a therapeutic agent. A known or a putative antibiotic is a chemical compound or formulation known and/or used as an antibiotic, or a chemical compound or formulation being investigated for use as an antibiotic. Likewise, a known or a putative toxic compound is a chemical compound or formulation known and/or used as a toxic compound, or a chemical compound or formulation being investigated for use or its activity as a toxic compound.

In one application of the method, the test agent will be a putative growth-inhibiting substance, such as an antimicrobial agent or a cytotoxic drug. The nature of the test agent, however, is virtually unlimited. The test agent could be, for example, any element, compound, mixture, drug, or putative drug, in any form (such as solid, liquid, or gas) or a set of environmental conditions (such as any combination of heat, humidity, light, oxygen level, and the like) desired to be tested, for example, for its cytotoxic effects, effects on metabolic activity or effects on cell proliferation. The method can also be used to test known antimicrobial agents and cytotoxic drugs for the purpose of determining which chemotherapeutic agents would be most effective against a given infection or cell type. The method can also be utilized with unknown or suspected antimicrobial agents and drugs for the purposes of determining their potential activity against a given microorganism or cell type, e.g., high-throughput screening of substances for biological activity as part of drug screening or other activities.

The assay methods and compositions provided herein can be used to, for example, assess viability, metabolic activity, proliferation and/or the effects of compounds on these qualities of a wide variety of living cells. Cells to be used in the methods may be collected from any source, and may be eukaryotic or prokaryotic. The cells may be primary cells, cultured cells or cell lines, including immortalized cell lines. The cells may be bacterial cells, fungal cells, plant cells, or animal cells. The cells may be vertebrate cells including, without limitation, mammalian cells such as primate cells, murine cells, and canine cells, or may be invertebrate cells including, without limitation, insect cells.

For example, human cells may be collected by medical personnel in their offices or clinics and sent to a central testing laboratory for testing using the present method, or cell specimens may be collected from patients in a hospital. The microbial specimens may come from any part of the human or animal body, such as, for example, from cerebral spinal fluid, an abscess, an infected wound, and genital infections. The cells may be from tumor biopsies or other specimens. The cells to be tested may also come from food samples, soil samples, or the like. The nature of the cells to be tested and their source is not critical to the invention.

The collected specimens are cultured on or in a suitable medium, as noted above, in accordance with conventional laboratory practice. From the microbial colonies or cellular clones on the primary culture plate, an inoculum is prepared in accordance with an established procedure which produces a microbial or cellular suspension of a prearranged concentration. Further processing of the suspension depends on the particular apparatus and method to be used for susceptibility testing.

One exemplary outcome of bacterial testing is to generate information on the probable success of treating a given population with a selected antibiotic. Another use of the methods and compositions provided herein is to test for the presence of microbes in a mixture, solution or on a surface thought to be free of them. The purpose of cellular cytotoxicity testing may be, for example, to determine the susceptibility of the tumor cells to particular chemotherapeutic drugs or test agents, or for screening potential drug candidates, or to determine whether a given drug candidate exhibits undesirable cytotoxicity to normal cells or any indicator cell type.

Exemplary cells that can be assessed with the methods and compositions provided herein include suspension cells, including without limitation Jurkat cells, adherent cells including without limitation NIH 3T3 cells, CHO-K1 cells, human dermal fibroblasts, and U-2 OS cells, and stem cells.

Methods described herein are inherently quantitative in that the amount of the reduced form of resazurin produced is proportional to the number of metabolically active cells. Accordingly, methods with the reagent solution can provide a quantitative measure of viability, proliferation and/or cytotoxicity. The methods, however, can be used both for quantitative testing and qualitative testing. The term qualitative testing refers to testing apparatus and methods which produce test results that generally indicate whether a microorganism, a cell or a population of cells is sensitive or resistant to a particular antibiotic or cytotoxic test agent. The relative degree of sensitivity or resistance is not reported in qualitative testing. The term quantitative testing refers to testing apparatus and methods which produce test results that provide data on the concentration of the antimicrobial, cytotoxic test agent, or other test agent that will be sufficient to inhibit growth or metabolic activity of the microorganism or other cell type. Typically, for microorganism specimens, six or more different dilutions of the test agent are utilized, covering the therapeutic range of concentrations of the test agent. The term minimum inhibitory concentration is often used to refer to the result provided by quantitative testing of a test agent and is defined as the minimum concentration of the test agent that will produce inhibition of the growth of the cells used in the method.

In certain embodiments, the fluorescence of the reduced form of resazurin, resorufin, is recorded. The amount of fluorescence is indicative of the quantity of metabolic activity, and thus cell viability and/or cell number present in the sample. In general, the resorufin is detected with excitation from about 540 nm to about 570 nm and emission from about 580 nm to about 610 nm. The exemplary filter set for fluorescent recording measures excitation at 560 nm and emission at 590 nm. For example, measuring fluorescence (monochrometer) excitation is at 560 nm (10 nm bandwidth) and emission at 590 nm (10 nm bandwidth. For another example, measuring fluorescence (filter) excitation is at 535 nm (25 nm bandwidth) and emission is at 615 nm (10 nm bandwidth). Exemplary measuring absorbance, excitation is at 570 nm and emission is at 600 nm (reference wavelength for normalization). Other slight variations from these wavelengths are possible.

If fluorescence is the endpoint measured, the vessel in which the reaction is performed, such as microtiter assay plate, should be compatible with a micro-well fluorimeter. If absorbance is the endpoint measure, the vessel in which the reaction is performed should be compatible with 570 nm absorbance measurements.

The methods provided herein are based on the use of a reagent solution, for example, to monitor and/or measure metabolic activity, viability or proliferation of a cell or cell culture population. In certain embodiments of the invention, the cells are incubated with the reagent solution at room temperature prior to measurement of reduced resazurin. In some embodiments, the cells are incubated with the reagent solution at 37° C. prior to measurement of reduced resazurin. In the rapid assay format of the methods, the incubation time of the cells with the reagent solution is less than 60 minutes. Typically, time of incubation of the cells with the reagent solution is from about 5 minutes to about 50 minutes. For example, the time of incubation of the cells with the reagent solution before the fluorescent or colorimetric data is measured is about 5 minutes, about 10 minutes, about 15 minutes, about 20 minutes, about 25 minutes, about 30 minutes, about 35 minutes, about 40 minutes, about 45 minutes, about 50 minutes, or about 55 minutes. In some embodiments, the time of incubation of the cells with the reagent solution before the fluorescent or colorimetric data is measured is from about 5 minutes to about 15 minutes, from about 10 minutes to about 30 minutes, or from about 15 minutes to about 30 minutes.

These incubation times may vary depending on, for example, the assay performed, the vessel in which the reaction is performed, the cell number or cell density, and/or whether absorbance or fluorescence measurements are made. For example, an exemplary incubation time of cells with the reagent solution before measuring bottom-read fluorescence is about 10 minutes, an exemplary incubation time of cells with the reagent solution before measuring top-read fluorescence is about 30 minutes, and an exemplary incubation time of cells with the reagent solution before measuring absorbance is about 20 minutes. An exemplary incubation time of cells with the reagent solution for use with a low cell number (for example, less than 5000 cells/100 microliters) is about 20 minutes.

In one aspect, reagent solution compositions for use in these methods are provided herein. Reagent solutions comprise resazurin, a buffer and an aqueous solvent. Exemplary aqueous solvents include, but are not limited to, water. In some embodiments, resazurin includes modified resazurin. In some embodiments, the resazurin is not a modified resazurin.

In some embodiments, the reagent solution does not comprise a chemically modified resazurin. In some embodiments, resazurin does not include modifications which increase its binding to cell membranes. In some embodiments, the reagent solution does not comprise C₁₂-resazurin.

The amount of resazurin in the reagent solution is sufficient to result in a measurable signal within 60 minutes of addition of the reagent solution to the cells. Typically, the amount of resazurin in the reagent solution is from about 0.25 mM to about 0.75 mM. For example, resazurin in the reagent solution is about 0.25 mM, about 0.30 mM, about 0.35 mM, about 0.40 mM, about 0.45 mM, about 0.50 mM, about 0.55 mM, about 0.60 mM, about 0.65 mM, about 0.70 mM, or about 0.75 mM. In some embodiments, resazurin in the reagent solution is from about 0.35 mM to about 0.60 mM. In some instances, resazurin in the reagent solution is about 0.38 mM, about 0.43 mM, about 0.48 mM, about 0.53 mM, or about 0.57 mM.

Typically, buffers with high buffering capacity within a physiological pH range for cells are suitable for use in the reagent solutions. Accordingly, suitable buffers include, but are not limited to, phosphate buffer (e.g., sodium or potassium), Tris buffers, HEPES, MOPS, ACES, PIPES, MOPSO, Bis-Tris propane, BES, TES, DIPSO, MOBS, TAPSO, Trizma, and HEPPSO.

The reagent solution should have a pH generally within a physiological pH range suitable for the cells to be assayed. The pH should also be suitable to provide a measurable signal within 60 minutes of addition of the reagent solution to the cells. Typically, the pH of the reagent solution is from about 7.0 to about 8.0. For example, the pH of the reagent solution is about 7.0, about 7.1, about 7.2, about 7.3, about 7.4, about 7.5, about 7.6, about 7.7, about 7.8, or about 7.9. In some embodiments, the pH range of the reagent solution is from about 7.2 to about 7.7. In some embodiments, the pH range of the reagent solution is from about 7.0 to about 7.5. In some embodiments, the pH of the reagent solution is about 7.4.

Buffer concentration of the reagent solution is such to result in a measurable signal within 60 minutes of addition of the reagent solution to the cells. Typically, the buffer concentration of the reagent solution is from about 0.05 M to about 1.0 M. For example, the buffer concentration of the reagent solution is about 0.08 M, about 0.09 M, about 0.10 M, about 0.20 M, about 0.3 M, about 0.40 M, about 0.50 M, about 0.6 M, about 0.7 M, about 0.80 M, about 0.90 M, or about 1.0 M. In some instances, the buffer concentration of the reagent solution is about 0.15 M, about 0.25 M, or about 0.75 M. In certain embodiments, the buffer concentration of the reagent solution is from about 0.09 M to about 0.3 M, from about 0.3M to about 1.0 M, or from about 0.09 M to about 1.0 M.

Exemplary reagent solutions contain an aqueous solvent, from about 0.35 mM to about 0.60 mM resazurin, and about 0.1 M to about 0.3 M phosphate buffer at a pH from about 7.2 to about 7.5. In some embodiments, the reagent solution consists essentially of an aqueous solvent, from about 0.35 mM to about 0.60 mM resazurin, and about 0.1 M to about 0.3 M phosphate buffer at a pH from about 7.2 to about 7.5. In certain embodiments, the reagent solution consists essentially of an aqueous solvent, about 0.48 mM resazurin, and about 0.15 phosphate buffer at pH 7.4. Reagent solutions provided herein do not include a poising agent, such as ferricyanide/ferrocyanide, as defined in U.S. Pat. No. 5,501,959.

It is preferred that the reagent solution be present in the form of a pre-made, ready to use reagent solution. In some embodiments, a ready-to-use reagent solution is preferably a 10× solution, that is, a volume equivalent to about one tenth of the final volume is added to a volume of cells plus media equivalent to about nine tenths of the final volume. For example, 100 microliters of reagent solution is added to 900 microliters of cells plus media in a cuvette and, for a 96-well plate assay, 10 microliters of reagent solution is added to 90 microliters of cells plus media. The preferred reagent solution is stable at room temperature (e.g., about 25° C.), refrigerated (e.g., about 4° C.) or frozen (e.g., about −20° C.). The kit, however, may also be configured so that the dye reagent is in the form of a lyophilized powder that is then reconstituted with an aqueous solvent (e.g., water) to yield a reagent solution that is non-toxic to cells.

Also provided herein are kits that contain reagent solution and instructions necessary to carry out the methods provided herein. In some embodiments, the kit includes a reagent solution comprising resazurin, a phosphate buffer and an aqueous solvent (wherein the dye is present in the solution in primarily the oxidized state) disposed in a container. In certain embodiments, the kit includes a reagent solution consisting essentially of an aqueous solvent, from about 0.35 mM to about 0.60 mM resazurin, and about 0.1 M to about 0.3 M phosphate buffer at a pH from about 7.2 to about 7.5, disposed in a container. The kit should also include instructions for how to use the reagent solution in a rapid assay format.

The following examples are provided for illustrative purposes only. The examples illustrate various aspects of the invention and practice of the methods of the invention. The examples are not intended to limit or define the entire scope of this invention and they are not intended to provide an exhaustive description of the many different embodiments of the invention. The reagents employed in the embodiments below are commercially available or can be prepared using commercially available instrumentation, methods, or reagents known in the art.

### EXAMPLES

### Example 1

Performance of the reagent solutions disclosed in this specification was compared in viability assays to commercially available indicator dyes.

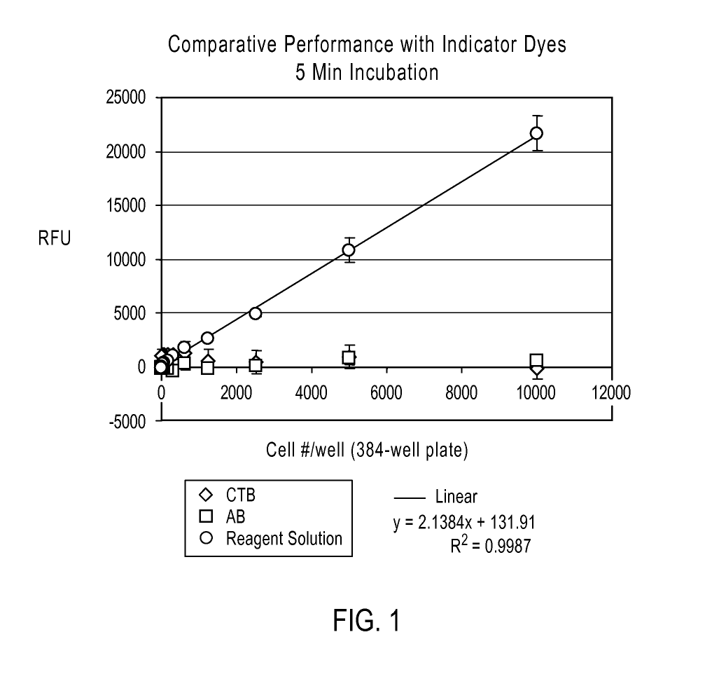

Cells from the adherent cell line CHO-K1 were plated in Dulbecco's Modified Eagle's Medium (D-MEM), 10% dialyzed fetal bovine serum (dFBS), 0.1 mM nonessential amino acids (NEAA), 25 mM HEPES, 100 U/ml penicillin, and 0.1 mg/ml streptomycin in 384-well plates at various cell densities and incubated at 37° C. and 5% CO₂. After 16-24 hours, reagent solution was added to the cells at a ratio of 1 part reagent to 9 parts culture media with cells (i.e, per well of a 384-well plate, 3.3 microliters of reagent solution was added to 30 microliters of cells+media). In other wells, the cells were loaded with the manufacturer's recommended amount of alamarBlue® dye (“AB”; Life Technologies Corporation), i.e., per well of a 384-well plate, 3.3 microliters of AB was added to 30 microliters of cells+media, or CellTiter-Blue® dye (“CTB”; Promega Corporation), i.e., per well of a 384-well plate, 6 microliters of CTB was added to 30 microliters of cells+media. After 5 minutes of incubation at 37° C. with the dyes, fluorescence in the wells was measured using a Tecan Safire 2 plate reader with excitation at 560 nm and emission at 590 nm. FIG. 1 shows the comparative performance of the indicator dyes following the 5 minute incubation with the dyes. The assay performed with a 5 minute incubation of the cells with the reagent solution disclosed herein provided viability data for cells at various densities. Neither CTB nor AB provided a detectable signal after a 5 minute incubation. Instructions for use accompanying CTB and AB recommend a 1-4 hour incubation time with the dye solution before reading the results.

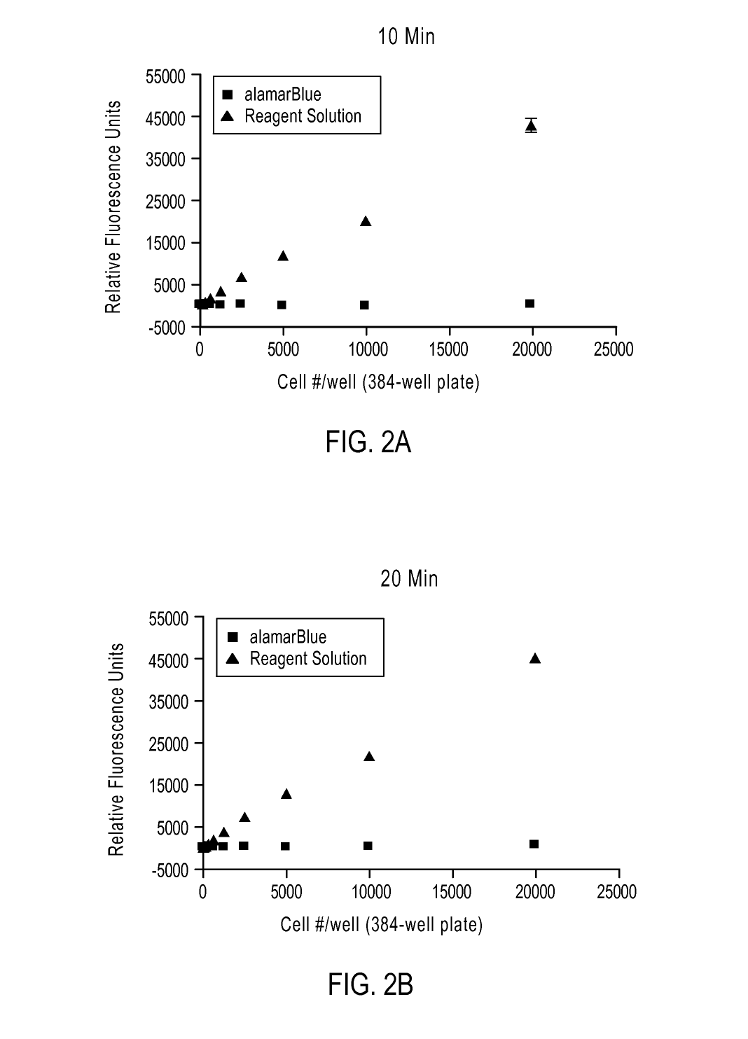

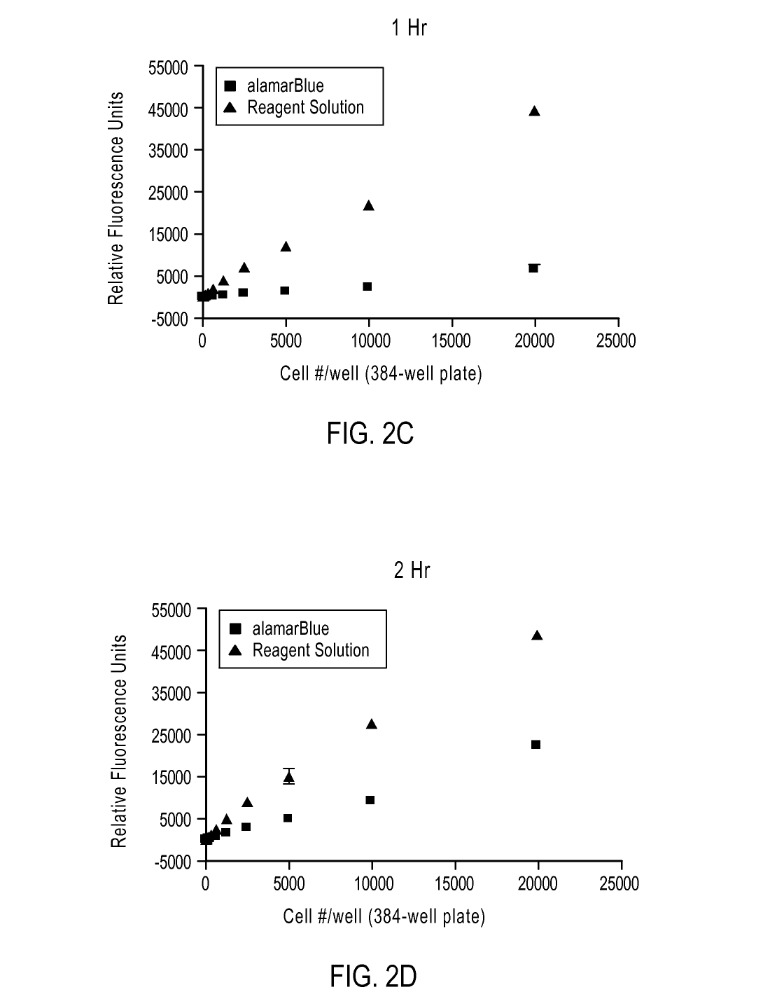

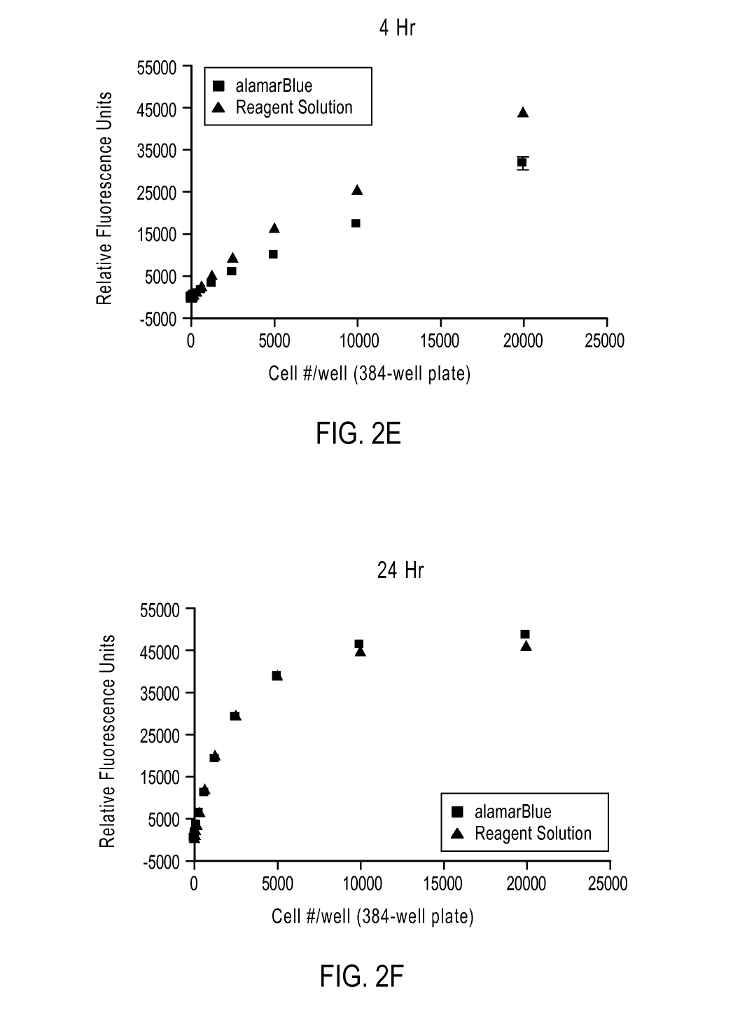

Viability assays using various dye incubation times were performed with the reagent solutions and alamarBlue® (“AB”). CHO-k1 cells were plated at various cell densities as described above. After 16-24 hours, cells were loaded with the reagent solution or AB as described above and the cells were incubated with the dyes for varying times at 37° C. Following incubation, fluorescence was measured using a Tecan Safire 2 plate reader with excitation at 560 nm and emission at 590 nm. FIGS. 2A-2F show the comparative performance of the dyes with a 10 min, 20 min, 1 hr, 2 hr, 4 hr, or 24 hr incubation time. The reagent solution provided clear viability data at all incubation times tested and at all cell densities tested. The reagent solution provided clear viability data after a 10 min incubation whereas AB required at least a 2 hr incubation before fluorescent signal was clearly detectable at the various cell densities. After incubations between 10 min and 4 hrs, the reagent solution described herein resulted in a greater relative fluorescence signal than AB. The signal to background ratio is also larger for the reagent solution compared to commercially available resazurin-based products for incubation time of 2-4 hours or less (exemplified in FIG. 2).

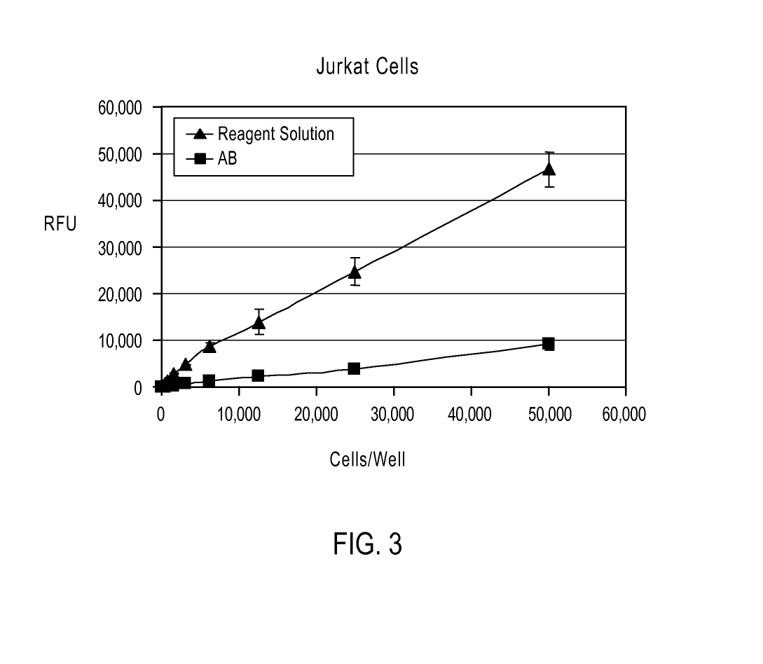

In another assay, Jurkat cells, which are grown in suspension, were plated at various cell densities in RPMI supplemented with 10% dFBS, 0.1 mM NEAA, 25 mM HEPES, 100 U/ml penicillin, and 0.1 mg/ml streptomycinin 384-well plates. Reagent solution or AB was added to the cells at a ratio of 1 part reagent to 9 parts culture media with cells (i.e, per well of a 384-well plate, 4 microliters of reagent solution or AB was added to 36 microliters of cells) and, after 13 minutes of incubation at 37° C., fluorescence in the wells was measured using a Tecan Safire 2 plate reader with excitation at 560 nm and emission at 590 nm. The results are shown in FIG. 3 with the relative fluorescence values plotted versus cells per well. With Jurkat cells, a suspension cell line, the reagent solution described herein resulted in a clear viability result and a greater relative fluorescence signal than AB in a short incubation assay format.

### Example 2

Reagent solutions containing resazurin in a variety of buffers, at varying buffer concentrations, at varying resazurin concentrations, and at varying pH values were examined in cell viability assays.

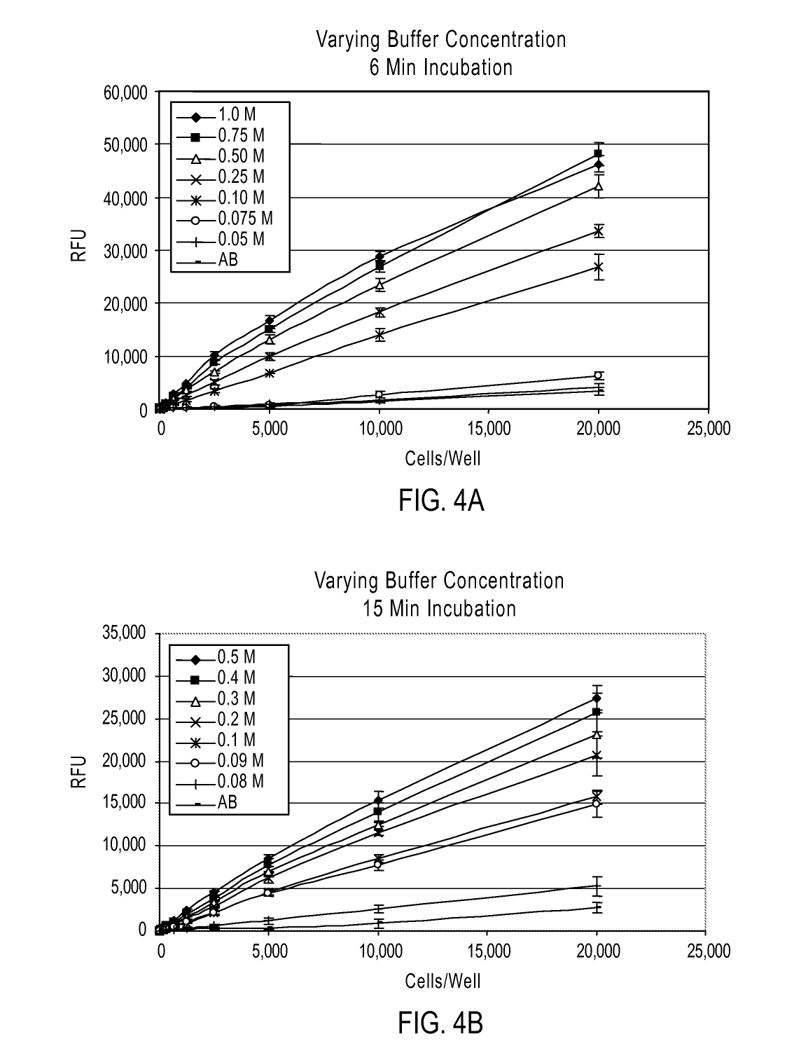

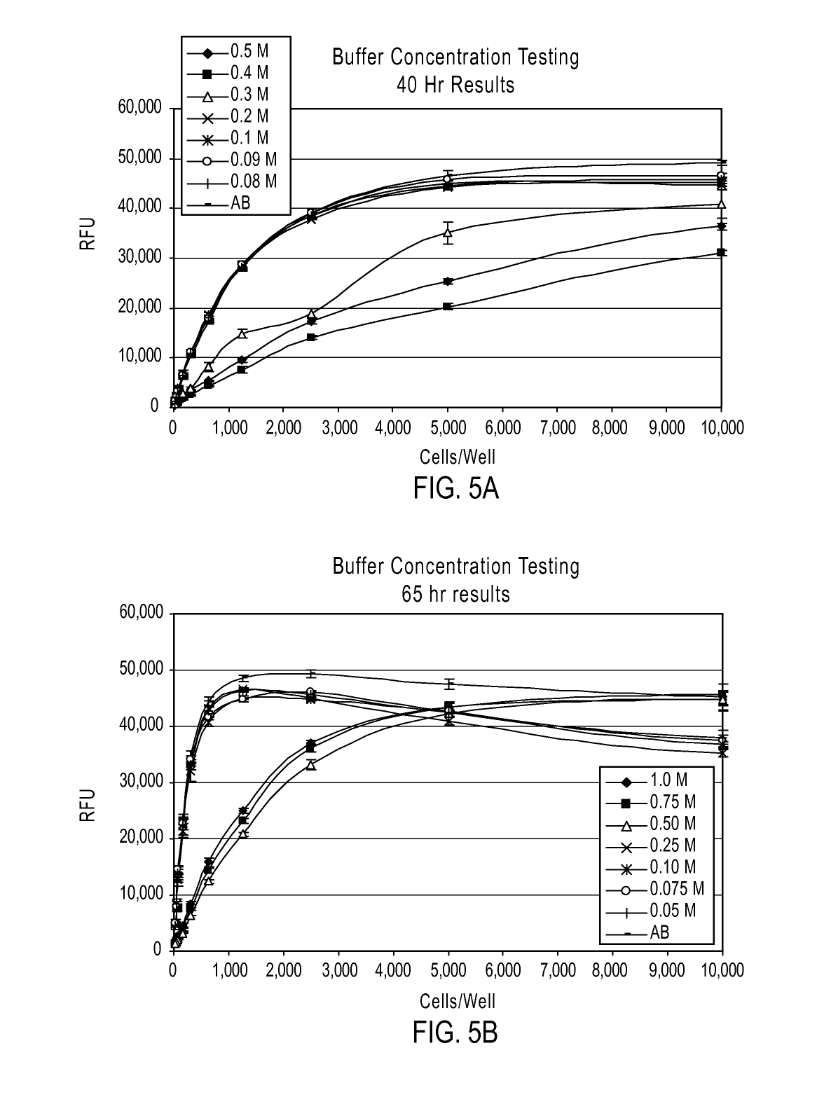

For example, various concentrations of phosphate buffer were tested in the reagent solution with the resazurin concentration at 0.48 mM. These phosphate buffer concentrations included 1.0 M, 0.75 M, 0.5 M, 0.4 M, 0.3 M, 0.25 M, 0.2 M, 0.1 M, 0.09 M, 0.08 M, 0.075 M, and 0.05 M, all pH 7.4. CHO-K1 cells were plated in D-MEM with 10% dFBS, 0.1 mM NEAA, 25 mM HEPES, 100 U/ml penicillin, and 0.1 mg/ml streptomycin in 384-well plates at various cell densities and incubated at 37° C. and 5% CO₂. After 16-24 hours, reagent solution (10×) was added to the cells at a ratio of 1 part reagent to 9 parts culture media with cells, or alamarBlue® (“AB”; 10×) was added to the cells. Following incubation at 37° C., fluorescence was measured using a Tecan Safire 2 plate reader with excitation at 560 nm and emission at 590 nm. FIGS. 4A and 5B show performance of the reagent solutions with phosphate buffer ranging from 1.0 M-0.05 M with a 6 min and 65 hr incubation time, respectively. FIGS. 4B and 5A show performance of the reagent solutions with phosphate buffer ranging from 0.5 M-0.08 M with a 15 min and 40 hr incubation time, respectively. As shown in FIGS. 4A and 4B, buffer concentrations of 0.09 M and above led to reagent solutions suitable for the rapid assay format described herein and exhibited a significant performance advantage over AB for short incubation time points. Buffer concentrations up to 1.0 M resulted in reagent solutions that performed well in short incubation time assays, e.g., 6 min, 10 min, 15 min incubation times. With buffer concentrations at 0.3 M or above used in extended incubation times (e.g., 40 hr, 65 hr incubation times), signs of cell toxicity were observed.

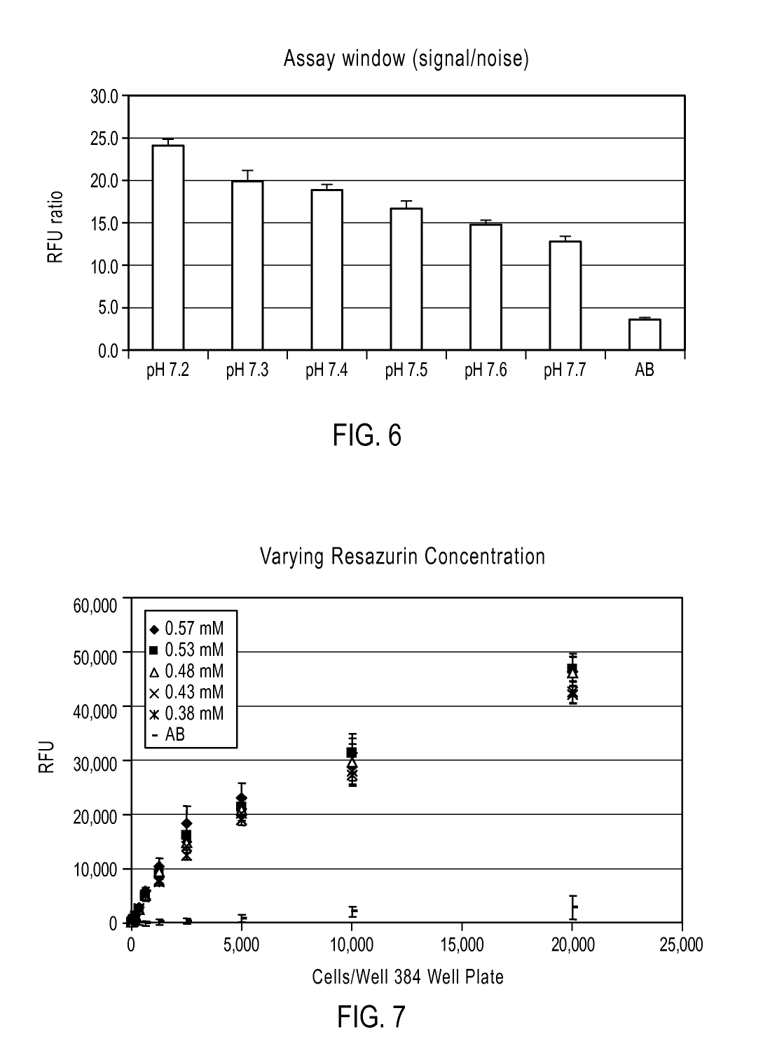

The effects of the pH of the phosphate buffer on assay performance were also assessed. For this test, the performance of 0.48 mM resazurin in 0.15 M phosphate buffer at pH 7.2, 7.3, 7.4, 7.5, 7.6, 7.7 was tested. CHO-K1 cells were plated at 5000 cells/well in 384-well plates in the culture medium described above and incubated at 37° C. and 5% CO₂. After 16-24 hours, reagent solution or AB was added to the cells at a ratio of 1 part reagent to 9 parts culture media with cells. Following incubation at 37° C. for 10 minutes, fluorescence was measured using a Tecan Safire 2 plate reader with excitation at 560 nm and emission at 590 nm. FIG. 6 shows the ratio of relative fluorescence units (RFU) measured in wells with cells to RFU of control wells with no cells. As shown in FIG. 6, the assay window increased with decreasing pH, although all pH values tested provided a reagent solution that performed well in a rapid assay format. The highest assay windows, i.e., signal/background, were observed at pH 7.2.

The effect of varying the concentration of resazurin in the reagent solution was also tested. For this test, resazurin solutions at 0.38, 0.43, 0.48, 0.53, or 0.57 mM in 0.15 M phosphate buffer, pH 7.4 were created. Cells from the immortalized, adherent cell line NIH 3T3 were plated at various densities in D-MEM supplemented with 10% newborn calf serum, 0.1 mM NEAA, 25 mM HEPES, 100 U/mL penicillin, and 0.1 mg/mL streptomycin in 384-well plates and incubated at 37° C. and 5% CO₂. After 16-24 hours, reagent solution or AB was added to the cells at a ratio of 1 part dye to 9 parts culture media with cells. Following incubation at 37° C. for 13 minutes, fluorescence was measured using a Tecan Safire 2 plate reader with excitation at 560 nm and emission at 590 nm. As shown in FIG. 7, all of these reagent solution formulations performed well in the rapid assay format and showed a significant improvement over AB.

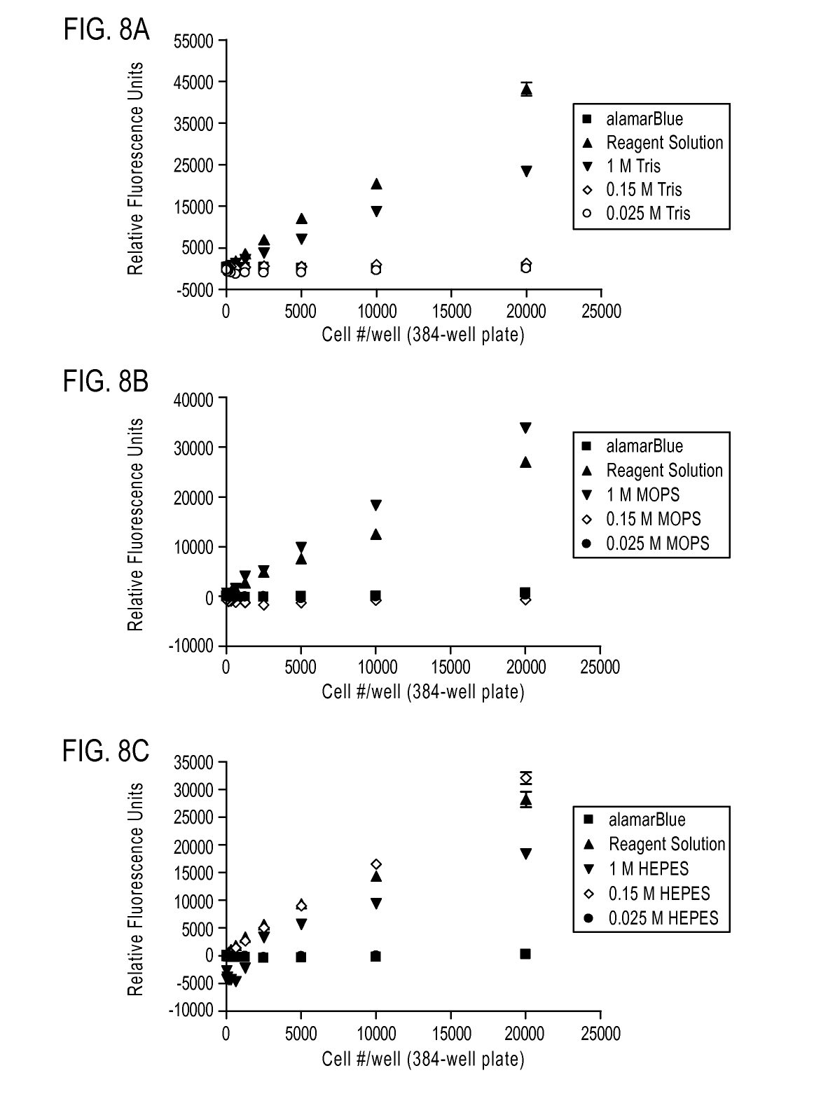

The effect of buffer composition of the reagent solution on assay performance was also assessed. For this test, several representative buffers (Tris, MOPS, and HEPES) with high buffering capacity in the pH range of 7.0 to 7.5 were selected. Reagent solutions were made with 0.48 mM resazurin in 1 M, 0.15 M, or 0.025 M Tris, MOPS, or HEPES buffer, at pH 7.4. The performance of each solution was compared to a standard reagent solution formulation (0.48 mM resazurin in 0.15 M phosphate buffer, pH 7.4) and to AB. CHO-K1 cells were plated at various densities in the medium described above in 384-well plates and incubated at 37° C. and 5% CO₂. After 16-24 hours, reagent solution or AB was added to the cells at a ratio of 1 part dye to 9 parts culture media with cells. Following incubation at 37° C. for 10 minutes, fluorescence was measured using a Tecan Safire 2 plate reader with excitation at 560 nm and emission at 590 nm. As shown in FIG. 8, all three buffers (Tris, MOPS, and HEPES) at 1 M concentration produced signals similar to the standard reagent solution at 10 minutes. Furthermore, the 1M concentration for these three buffers (Tris, MOPS, and HEPES) did not appear to induce cell lysis. Of the 0.15 M buffers, only HEPES produced a signal similar to that of the standard reagent solution at 10 minutes. None of the buffer formulations at 0.025 M produced a signal similar to the standard reagent solution.

### Example 3

Performance of the reagent solutions disclosed in this specification was compared in cell cytoxicity assays to commercially available indicator dyes.

Cells from the adherent human cell line U-2 OS were plated in McCoy's 5A Medium supplemented with 10% dFBS, 0.1 mM NEAA, 100 U/mL penicillin, 0.1 mg/mL streptomycin, 1 mM sodium pyruvate, and 25 mM HEPES at 2000 cells/well in 384-well plates. The cells were then exposed to various concentration of Etoposide, Doxorubicin, Chetomin or Staurosporine for 72 hours at 37° C., 5% CO₂. In some wells, reagent solution was added to the cells at a ratio of 1 part reagent to 9 parts culture media with cells. In other wells, the cells were loaded with the manufacturer's recommended amount of alamarBlue® dye (Life Technologies Corporation), CellTiter-Blue® dye (Promega Corporation), or CellTiter-Glo® dye (Promega Corporation). After 10 minutes of incubation at 37° C. with the dyes, read-out signal from the wells was measured. For wells with reagent solution, alamarBlue®, and CellTiter-Blue®, fluorescence was measured and for wells with CellTiter-Glo®, luminescence was measured.

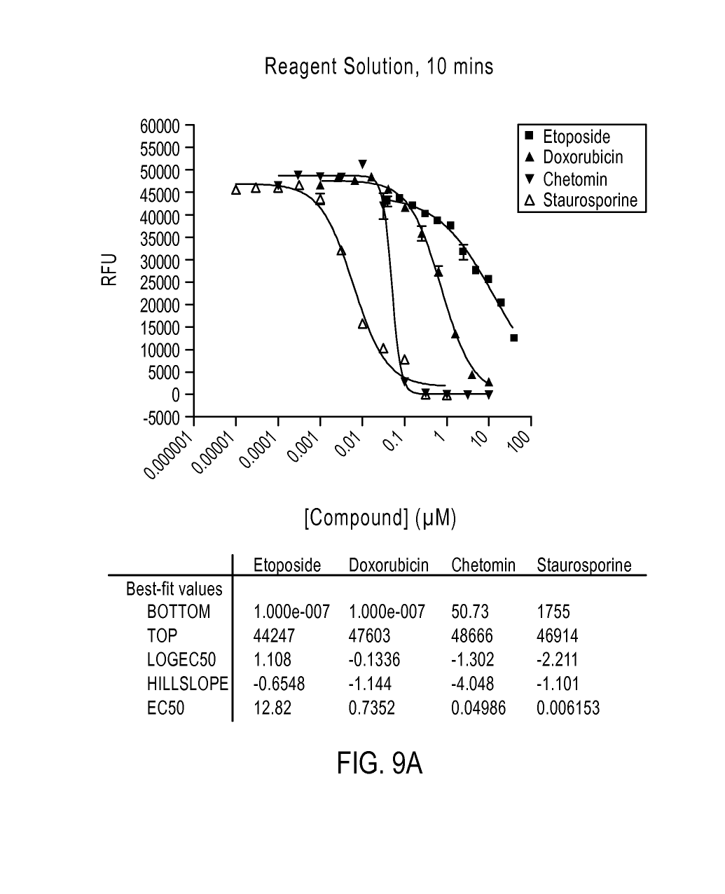

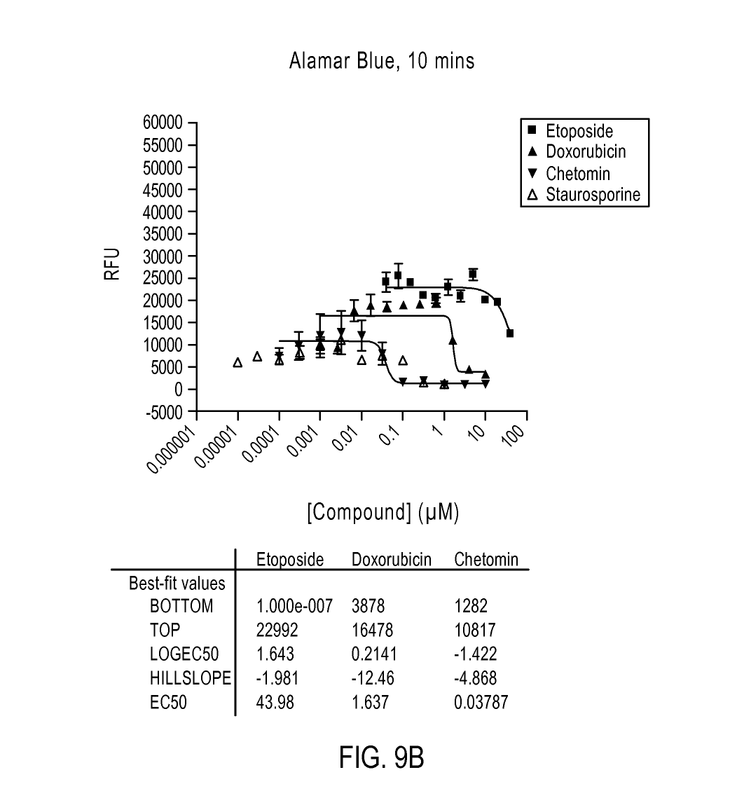

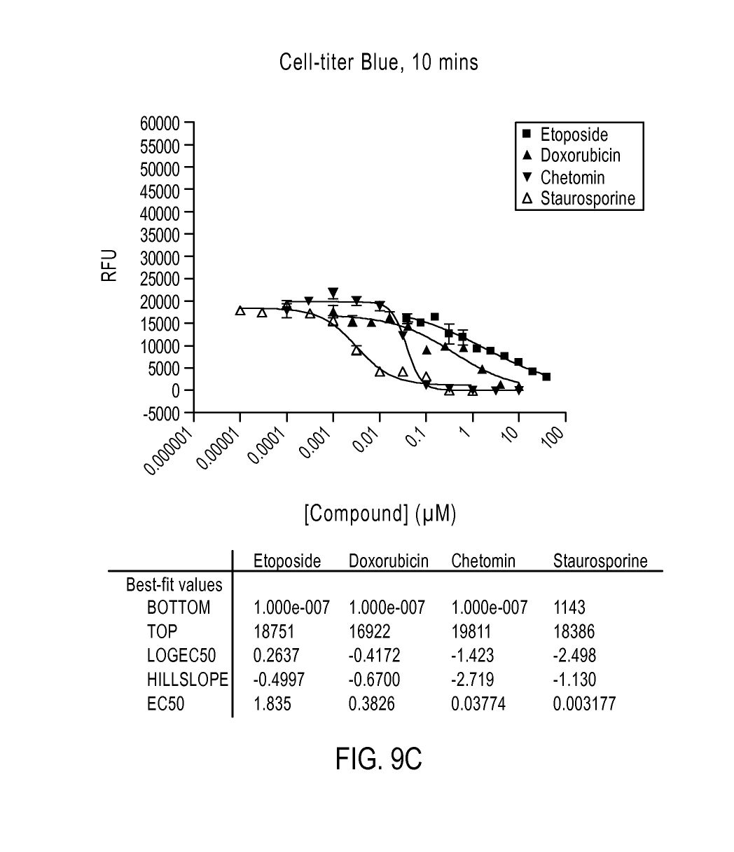

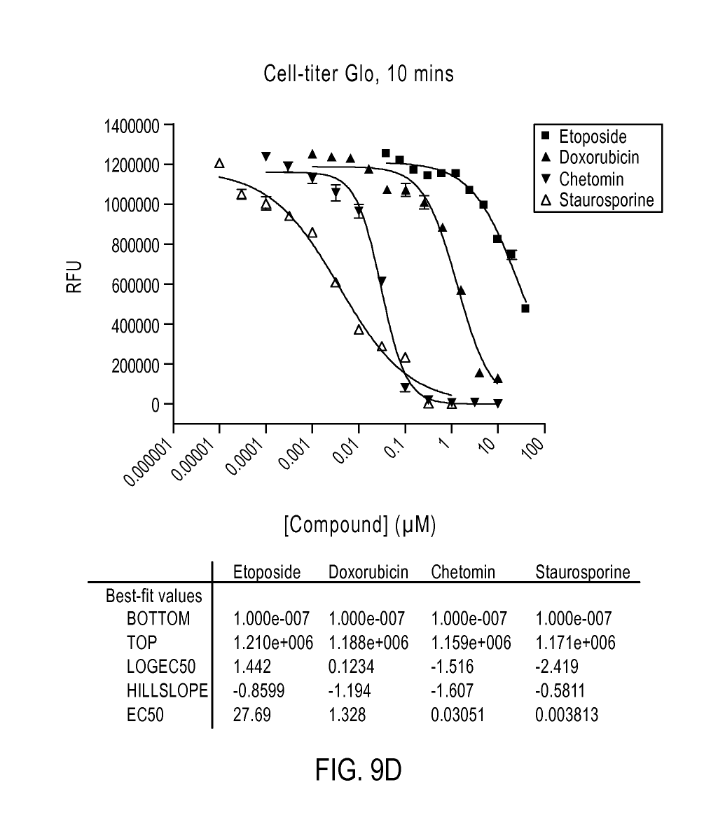

FIG. 9 shows the results with each cytotoxicity assay. The cytotoxicity assays with the reagent solution as disclosed herein provided clear results consistent with that known for these compounds with cytotoxic acticity. Compared to the other resazurin-based dyes in the rapid assay format (10 min incubation time), the read-out signal provided by the reagent solution is much better for generation of an EC50 value for the test compounds. The EC50 values generated using the reagent solution are similar to those generated with CellTiter-Glo, a luciferase/luciferin based assay which requires cell lysis to generate the signal.

### Example 4

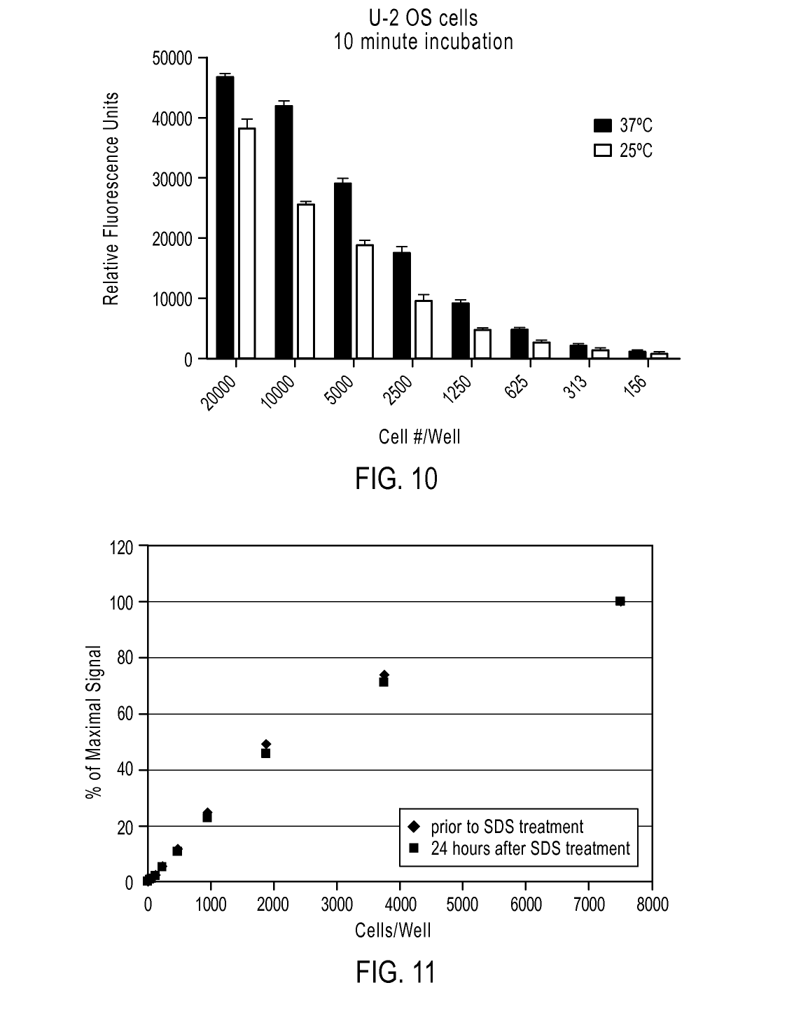

The effect of temperature during the incubation the reagent solution with cells at varying cell densities was assessed. U-2 OS cells were plated in D-MEM supplemented with 10% dFBS, 0.1 mM NEAA, 25 mM HEPES, 100 U/ml penicillin, and 0.1 mg/ml streptomycin in 384-well plates at densities of 156, 313, 625, 1250, 2500, 5000, 10,000, and 20,000 cells per well and incubated at 37° C. and 5% CO₂. After 20 hours, reagent solution was added to the cells at a ratio of 1 part reagent to 9 parts culture media with cells and the cells were incubated for 10 minutes at 25° C. or at 37° C. with 5% CO₂. Fluorescence in the wells was measured using a Tecan Safire 2 Plate reader (Excitation of 560 nm, Emission of 590 nm). As shown in FIG. 10, a correlation between increasing fluorescence values and increasing cell densities was observed for either temperature incubation.

Stability of the signal from the assay and reagent solution disclosed herein was assessed. Primary human dermal fibroblast cells were plated at various cell densities per well in Medium 106 supplemented with Low Serum Growth Supplement (2% fetal bovine serum, 1 microgram/ml hydrocortisone, 10 ng/ml human epidermal growth factor, 3 ng/ml basic fibroblast growth factor, 10 microgram/ml heparin), 10 microgram/ml gentamicin, and 0.25 microgram/ml amphotericin B in a 384-well plate and incubated at 37° C. and 5% CO₂. After 16-24 hours, reagent solution was added to cells at a ratio of 1 part reagent to 9 parts culture media with cells and the cells were incubated for 6 hours at 37° C. and 5% CO₂. Fluorescence in the wells was measured using a Tecan Safire 2 plate reader (Excitation of 560 nm, Emission of 590 nm). Following this measurement, 20 microliters of 3% sodium dodecylsulfate (SDS) was added to each well giving a final concentration of 1% SDS in the well. The cells in the plate were incubated for 24 hour at room temperature prior to measuring the fluorescence again. As there was some dilution of the signal with the addition of the 3% SDS, values shown in FIG. 11 were normalized by taking the highest fluorescence values and setting these to 100% and the no cell control wells and setting these to 0%. Virtually identical values are observed for cells prior to SDS treatment and 24 hours after SDS treatment (FIG. 11).

### Example 5

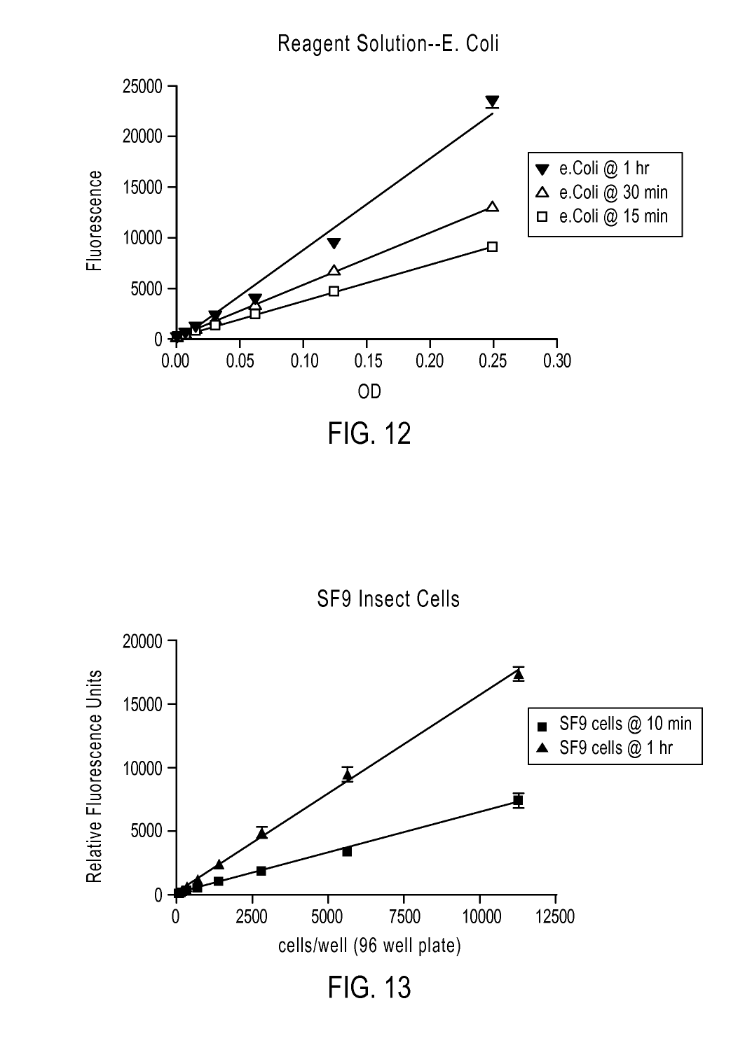

The reagent solutions are also suitable for assessment of bacterial and invertebrate cells in a rapid assay format. For example, E. coli cells were plated at various concentrations in LB medium in a 96-well plate. The cells were loaded with reagent solution at a ratio of 1 part reagent to 9 parts culture media with cells (i.e, per well of a 96-well plate, 10 microliters of reagent solution was added to 90 microliters of cells) and incubated at 37° C. for 15 minutes, 30 minutes, or 1 hour. The relative fluorescence was determined using a Tecan Safire2 plate reader (excitation 560 nm, emission 590nm). As shown in FIG. 12, there is a linear correlation between the number of bacterial cells in the well and the relative fluorescence observed.

The insect cells SF9 cells were plated at various concentrations in SF-900 III SFM Media supplemented with 5 mM L-glutamine and 0.5× Antibiotic-Antimycotic (Invitrogen Catalog No. 15240) in a 96-well plate. The cells were loaded with reagent solution at a ratio of 1 part reagent to 9 parts culture media with cells and incubated at 27° C. for 10 minutes or 1 hour. The relative fluorescence was determined using a Tecan Safire2 plate reader (excitation 560 nm, emission 590 nm). As shown in FIG. 13, there is a linear correlation between the number of insect cells in the well and the relative fluorescence observed.

Although the foregoing invention is described in some detail by way of illustration and example for purposes of clarity of understanding, those of ordinary skill in the art will realize readily that many changes and modifications can be made thereto without departing from the spirit or scope of the appended claims.

1. A method for assessing a metabolic activity of a cell in a cell culture, the method comprising adding a reagent solution comprising resazurin, a buffer and an aqueous solvent to the cell culture, wherein the resazurin is initially present in the oxidized state and the assessing of metabolic activity is performed by measuring for production of the reduced state of the resazurin within 60 minutes of adding the reagent solution to the cell culture.

2. The method of claim 1, wherein the measuring comprises fluorescent spectroscopy or visible spectroscopy.

3. The method of claim 1, wherein the measuring is performed without separating the culture medium from the cell.

4. The method of claim 1, wherein the adding of the reagent solution and the measuring for production of the reduced state of resazurin are both performed in the same vessel.

5. The method of claim 1, wherein the measuring is performed within 30 minutes of adding the reagent solution.

6. The method of claim 5, wherein the measuring is performed about 20 minutes after adding the reagent solution or about 10 minutes after adding the reagent solution.

7. (canceled)

8. The method of claim 1, wherein the cell is prokaryotic cell, an eukaryotic cell, a primary eukaryotic cell, a cultured eukaryotic cell, a mammalian cell, an insect cell or a bacterial cell.

9-14. (canceled)

15. The method of claim 1, wherein the cell is grown in suspension culture, monolayer culture, adherent culture or on an absorptive disk.

16. The method of claim 1, wherein the buffer is a phosphate buffer, a Tris buffer, HEPES, or MOPS.

17. The method of claim 1, wherein the reagent solution comprises about 0.1 M to about 0.3 M phosphate buffer.

18. The method of claim 1, wherein the reagent solution comprises from about 0.35 mM to about 0.60 mM resazurin.

19. The method of claim 1, wherein the reagent solution buffer has a pH of from about 7.0 to about 7.8.

20. The method of claim 1, wherein the reagent solution comprises: 0.35 mM to about 0.60 mM resazurin, and about 0.1 M to about 0.3 M phosphate buffer at a pH from about 7.2 to about 7.5.

21. The method of claim 1, wherein the cell is suitable for another functional assay following the assessing for metabolic activity.

22. A method for determining cytotoxicity of a test agent, the method comprising:
(a) contacting a living cell in a culture medium with the test agent and incubating the cell and the test agent in the culture medium for an amount of time;
(b) adding to the cell and the culture medium from (a) a reagent solution comprising resazurin, a buffer and an aqueous solvent, wherein the resazurin is initially present in an oxidized state; and
(c) measuring for production of a reduced state of the resazurin from (b) within 60 minutes of addition of the reagent solution, wherein the measuring indicates the cytotoxicity of the test agent.

23. The method of claim 22, wherein the measuring comprises fluorescent spectroscopy or visible spectroscopy.

24. The method of claim 22, wherein the measuring is performed without separating the culture medium from the cell.

25. The method of claim 22, wherein the adding of the reagent solution in (b) and the measuring in (c) are both performed in the same vessel.

26. The method of claim 22, wherein the contacting and the incubating in (a), the adding of the reagent solution in (b) and the measuring in (c) are all performed in the same vessel.

27. The method of claim 22, wherein the measuring is performed less than 30 minutes after the adding of the reagent solution in (b).

28. The method of claim 27, wherein the measuring is performed about 20 minutes after the adding of the reagent solution in (b) or about 10 minutes after the adding of the reagent solution in (b).

29. (canceled)

30. The method of claim 22, wherein the cell is a prokaryotic cell, a eukaryotic cell a cultured eukaryotic cell a mammalian cell an insect cell or a bacterial cell.

31.-36. (canceled)

37. The method of claim 22, wherein the cell is grown in a suspension culture, a monolayer culture, an adherent culture or on an absorptive disk.

38. The method of claim 22, wherein in the test agent is a known or a putative antibiotic, a known or a putative pharmaceutical or a therapeutic agent or a known or putative toxic compound.

39.-40. (canceled)

41. The method of claim 22, wherein the buffer is a phosphate buffer, a Tris buffer, HEPES, or MOPS.

42. The method of claim 22, wherein the reagent solution comprises about 0.1 M to about 0.3 M phosphate buffer.

43. The method of claim 22, wherein the reagent solution comprises from about 0.35 mM to about 0.6 mM resazurin.

44. The method of claim 22, wherein the reagent solution buffer has a pH of from about 7.0 to about 7.8.

45. The method of claim 22, wherein the reagent solution comprises: 0.35 mM to about 0.60 mM resazurin, and about 0.1 M to about 0.3 M phosphate buffer at a pH from about 7.2 to about 7.5.

46. The method of claim 22, wherein the cell is suitable for a functional assay following the measuring in (c).

47. A method for determining cytotoxicity of a test agent, the method comprising:
(a) contacting a living cell in a culture medium with a pre-determined amount of the test agent and incubating the cell and the test agent in the culture medium for a pre-determined amount of time;
(b) adding to the cell and the culture medium from (a) a reagent solution comprising resazurin, a buffer and an aqueous solvent; and
(c) measuring for production of resorufin within 60 minutes of addition of the reagent solution, wherein the measuring indicates cytotoxicity of the test agent.

48. The method of claim 47, wherein production of the resorufin is measured fluorimetrically or colorimetrically.

49. (canceled)

50. A method for determining cytotoxicity of a test agent, the method comprising:
(a) contacting a living cell in a culture medium with the test agent and incubating the cell and the test agent in the culture medium for an amount of time;
(b) adding to the cell and the culture medium from (a) a reagent solution consisting essentially of resazurin, a buffer and an aqueous solvent, wherein the resazurin is initially present in an oxidized state; and
(c) measuring for production of a reduced state of the resazurin from (b), wherein the measuring indicates cytotoxicity of the test agent.

51. A method for determining cytotoxic effect of a test agent on growth of cultured cells, the method comprising:
(a) culturing the cells in a growth medium in the presence of the test agent for an amount of time;
(b) adding to the cells and the growth medium from (a) a reagent solution comprising resazurin, a buffer and an aqueous solvent; and
(c) measuring for production of resorufin within 60 minutes of the addition of the reagent solution in (b), wherein the measuring indicates the cytotoxic effect of the test agent on growth of the cells.

52. A method for determining cytotoxicity of a test culture condition, the method comprising:
(a) culturing living cells in a culture medium under a test culture condition for an amount of time;
(b) adding to the cells and the culture medium from (a) a reagent solution comprising resazurin, a buffer and an aqueous solvent, wherein the resazurin is initially present in an oxidized state; and
(c) measuring for production of a reduced state of the resazurin from (b) within 60 minutes of the addition of the reagent solution, wherein the measuring indicates the cytotoxicity of the test culture condition.

53. A kit for assessing metabolic activity of a cell comprising a reagent solution disposed in a container, the reagent solution consisting essentially of an aqueous solvent, from about 0.35 mM to about 0.60 mM resazurin, and about 0.1 M to about 0.3 M phosphate buffer at a pH from about 7.2 to about 7.5.
